(19) United States
(12) Patent Application Publication
(10) Pub. No.: US 2020/0101404 A1
(43) Pub. Date: Apr. 2, 2020

(54) BIOLOGICAL COMPONENT COLLECTION KIT AND BLOOD COMPONENT COLLECTION SYSTEM

(71) Applicant: TERUMO KABUSHIKI KAISHA, Tokyo (JP)

(72) Inventor: Masatsugu IGARASHI, Shizuoka (JP)

(73) Assignee: TERUMO KABUSHIKI KAISHA, Tokyo (JP)

(21) Appl. No.: 16/364,269

(22) Filed: Mar. 26, 2019

(30) Foreign Application Priority Data
Mar. 26, 2018 (JP) 2018-057404

(51) Int. Cl. B01D 29/05; B01L 3/00; A61M 1/02; B01D 29/60

(52) U.S. Cl. CPC B01D 29/05; B01L 3/502; A61M 1/025; B01D 29/60; B01L 2300/12; A61M 2202/0415; B01L 2400/0481; B01L 2300/06; B01L 2200/0647; A61M 2202/0429; A61M 2202/0427; B01L 2300/0681

(57) ABSTRACT

A third cassette (28) of a blood component collection kit (12) is equipped with a third cassette main body (29) in which a flow path (F) is formed together with having a filter accommodating unit (86), and a filter member (88) disposed in the filter accommodating unit (86). The flow path (F) includes a red blood cell line (f1) in which red blood cells flow, a preservation solution line (f2) in which a preservation solution flows, a confluence section (f3) in which the red blood cells and a preservation solution are made to merge together, a mixed liquid line (f4) through which a mixed liquid of the red blood cells and the preservation solution flows, and a filter chamber (f5) formed inside the filter accommodating unit (86).

### TECHNICAL FIELD

The present invention relates to a blood component collection kit and a blood component collection system.

### BACKGROUND ART

Conventionally, a blood component collection system is known in which blood (whole blood) is collected from a donor, and a predetermined blood component is separated and recovered from the blood (for example, refer to Japanese Laid-Open Patent Publication No. 2013-514863 (PCT)). The blood component collection system is equipped with a separation device, and a blood component collection kit that is mounted in the separation device.

### SUMMARY OF INVENTION

Incidentally, from the standpoint of workability, it is preferable that the attachment of the blood component collection kit to the separation device can be carried out easily and efficiently. Further, from an economical standpoint, it is preferable for manufacturing costs of the blood component collection kit to be reduced. Furthermore, it is preferable that a filter for removing a predetermined component is provided in the blood component collection kit.

Thus, an object of the present invention is to provide a blood component collection kit and a blood component collection system, in which it is possible to easily and efficiently carry out attachment of the blood component collection kit to a separation device, wherein manufacturing costs can be reduced, and in which a predetermined component can be removed.

In order to achieve the aforementioned object, one aspect of the present invention is characterized by a blood component collection kit configured to be attachable to a separation device adapted to separate blood components from blood, comprising a sheet-shaped cassette main body which is formed by stacking two sheets made of a soft material, and in which a flow path is formed, together with having a filter accommodating unit, and a filter member disposed inside the filter accommodating unit, wherein the flow path includes a blood component line in which the blood components flow, a preservation solution line in which a preservation solution flows, a confluence section in which the blood components and the preservation solution are made to merge together, a mixed liquid line that communicates with the confluence section and through which a mixed liquid made up of the red blood cells and the preservation solution flows, and a filter chamber communicating with the mixed liquid line together with being formed inside the filter accommodating unit, the cassette main body comprises a flow path forming member adapted to form the flow path, in the flow path forming member, there is provided a pump action member made of a soft material that is pressed by a pump provided in the separation device, the flow path forming member includes a blood component line forming member adapted to form the blood component line, and a preservation solution line forming member adapted to form the preservation solution line, and the pump action member is disposed in at least one of the blood component line forming member and the preservation solution line forming member.

In accordance with the blood component collection kit, the blood components and the preservation solution are mixed in the cassette main body, and when the mixed liquid passes through the filter member, a predetermined component is removed from the mixed liquid, and a formulation from which the predetermined component has been removed is obtained. In accordance with such a blood component collection kit, the filter structure made up from the filter member and the filter accommodating unit is formed integrally with the cassette main body. Therefore, it is possible to reduce manufacturing costs of the blood component collection kit, and it is possible to efficiently carry out an operation to attach the blood component collection kit to the separation device. Further, since the pump action member is provided integrally with the cassette main body, it is possible to reduce manufacturing costs of the blood component collection kit provided with such a pump action member, and it is possible to efficiently carry out an operation of attaching the blood component collection kit with respect to a separation device equipped with a pump.

In the above-described blood component collection kit, the flow path forming member may include a blood component line forming member adapted to form the blood component line, and a preservation solution line forming member adapted to form the preservation solution line, and from among the blood component line forming member and the preservation solution line forming member, the pump action member may be disposed only in the preservation solution line forming member.

In accordance with such a configuration, since the number of pumps provided in the separation device is reduced, the configuration of the separation device can be simplified and costs can be reduced. Moreover, the amount at which red blood cells flow into the third cassette can be controlled by the blood collection speed or the like.

In the above-described blood component collection kit, the flow path forming member may include a mixed liquid line forming member adapted to form the mixed liquid line, and the pump action member may be disposed in the mixed liquid line forming member.

In accordance with such a configuration, since the number of pumps provided in the separation device is reduced, the configuration of the separation device can be simplified and costs can be reduced. By setting the ratio between the flow path cross-sectional area of the blood component line and the flow path cross-sectional area of the preservation solution line, it is possible to adjust a hematocrit value after confluence to a desired range.

In the above-described blood component collection kit, a portion of the blood component line adjacent to the confluence section, a portion of the preservation solution line adjacent to the confluence section, and the confluence section may be arranged on a straight line.

In accordance with such a configuration, the blood components and the preservation solution collide head-on at the confluence section. Therefore, mixing of the blood components and the preservation solution is promoted, and removal of the predetermined component can be efficiently performed by the filter member.

In the above-described blood component collection kit, the mixed liquid line may include a mixture promoting section the flow passage cross-sectional area of which varies along a direction of extension of the mixed liquid line.

In accordance with such a configuration, mixing of the blood components and the preservation solution is promoted, and removal of the predetermined component can be efficiently performed by the filter member.

In the above-described blood component collection kit, the filter accommodating unit may be constituted by a pressed portion made of a soft material which is pressed by a load detecting unit provided in the separation device in order to detect a pressure of the filter chamber.

Since the filter accommodating unit serves in a dual manner as the pressed portion for detecting the pressure, streamlining of the structure can be achieved.

Another aspect of the present invention is characterized by a blood component collection system equipped with a separation device adapted to separate blood components from blood, and a blood component collection kit configured to be attachable to the separation device, wherein the blood component collection kit comprises a sheet-shaped cassette main body which is formed by stacking two sheets made of a soft material, and in which a flow path is formed, together with having a filter accommodating unit, and a filter member disposed inside the filter accommodating unit, wherein the flow path includes a blood component line in which the blood components flow, a preservation solution line in which a preservation solution flows, a confluence section in which the blood components and the preservation solution are made to merge together, a mixed liquid line that communicates with the confluence section and through which a mixed liquid made up of the red blood cells and the preservation solution flows, and a filter chamber communicating with the mixed liquid line together with being formed inside the filter accommodating unit, the cassette main body comprises a flow path forming member adapted to form the flow path, in the flow path forming member, there is provided a pump action member made of a soft material that is pressed by a pump provided in the separation device, the flow path forming member includes a blood component line forming member adapted to form the blood component line, and a preservation solution line forming member adapted to form the preservation solution line, and the pump action member is disposed in at least one of the blood component line forming member and the preservation solution line forming member.

In the above-described blood component collection system, the separation device may comprise a load detecting unit, a pump, and a pump control unit adapted to control the pump, the filter accommodating unit may be constituted by a pressed portion made of a soft material which is pressed by the load detecting unit provided in the separation device in order to detect a pressure of the filter chamber, and the pump control unit may control the pump on the basis of a detection result of the load detecting unit, in a manner so that the pressure of the filter chamber falls within a predetermined range.

In accordance with such a configuration, while suppressing damage from occurring to the blood components which may be caused by excessive pressure, and by maintaining the pressure to be greater than or equal to a predetermined pressure, the ability to remove the predetermined component can be enhanced, and the process can be carried out efficiently.

In accordance with the blood component collection kit and the blood component collection system of the present invention, it is possible to reduce manufacturing costs, together with efficiently carrying out attachment of the blood component collection kit to the separation device.

### BRIEF DESCRIPTION OF DRAWINGS

FIG. 1 is a schematic diagram of a blood component collection system according to an embodiment of the present invention;

FIG. 2 is an explanatory diagram of a configuration of a third cassette;

FIG. 3 is a schematic cross-sectional view of the third cassette taken along line III-III in FIG. 2;

FIG. 4 is a schematic cross-sectional view of a filter structure of the third cassette;

FIG. 5 is a first explanatory diagram of operations of the blood component collection system;

FIG. 6 is a second explanatory diagram of operations of the blood component collection system;

FIG. 7 is a third explanatory diagram of operations of the blood component collection system;

FIG. 8 is a fourth explanatory diagram of operations of the blood component collection system;

FIG. 9 is a fifth explanatory diagram of operations of the blood component collection system;

FIG. 10 is a sixth explanatory diagram of operations of the blood component collection system;

FIG. 11 is a seventh explanatory diagram of operations of the blood component collection system;

FIG. 12 is an eighth explanatory diagram of operations of the blood component collection system;

FIG. 13 is a ninth explanatory diagram of operations of the blood component collection system;

FIG. 14A is an explanatory diagram of a configuration of the third cassette according to another embodiment and FIG. 14B is an explanatory diagram of a configuration of the third cassette according to yet another embodiment.

### DESCRIPTION OF EMBODIMENTS

Preferred embodiments of a blood component collection kit and a blood component collection system according to the present invention will be presented and described in detail below with reference to the accompanying drawings.

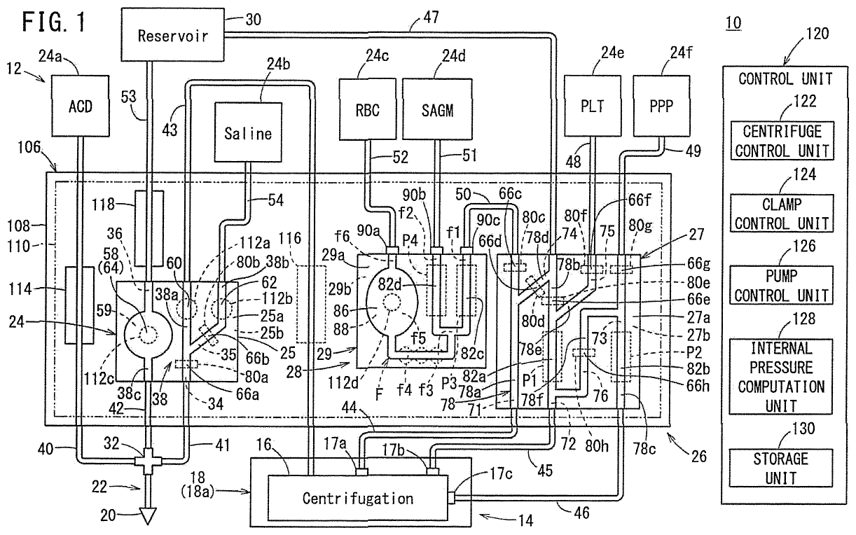

As shown in FIG. 1, the blood component collection system 10 according to the present embodiment is configured as a system in which, by collecting blood (whole blood) from a donor and subjecting the blood to centrifugal separation outside the body, at least one type of blood component is collected, and the remaining blood components are returned to the donor. According to the present embodiment, the blood component collection system 10 collects red blood cells (leukocyte-removed red blood cells: RBC), blood platelets (PLT), and blood plasma (platelet poor plasma: PPP).

The blood component collection system 10 is equipped with a blood component collection kit 12 (hereinafter referred to as a “collection kit 12”) for enabling storage and flow of blood components therein, and a centrifugal separation device 14 (one form of a separation device) that applies a centrifugal force to a predetermined portion (a blood treatment unit 16, to be described later) of the collection kit 12. The collection kit 12 includes a blood treatment unit 16 to which there is introduced whole blood that is removed from the donor, and the whole blood is centrifugally separated into a plurality of blood components. The centrifugal separation device 14 is equipped with a centrifuge unit 18 having a centrifugal rotor 18a for applying a centrifugal force to the blood treatment unit 16. The blood treatment unit 16 is capable of being mounted in the centrifuge unit 18.

The blood collection kit 12 is discarded every time that it is used in order to prevent contamination and ensure sanitation. The collection kit 12 comprises a blood collection and blood returning unit 22 having a blood collecting needle 20, the blood treatment unit 16, a first blood component collection cassette 24 (hereinafter referred to as a “first cassette 24”), a second blood component collection cassette 26 (hereinafter referred to as a “second cassette 26”), a third blood component collection cassette 28 (hereinafter referred to as a “third cassette 28”), and a reservoir 30.

The collection kit 12 further includes an ACD solution bag 24a containing an ACD solution which is an anticoagulant, a rinsing solution bag 24b containing physiological saline as a rinsing solution, a red blood cell bag 24c for storage of leukocyte-removed red blood cells (a red blood cell formulation from which leukocytes or white blood cells have been removed), a preservation solution bag 24d containing the preservation solution, a blood platelet bag 24e for storage of blood platelets, and a PPP bag 24f for storage of blood plasma (platelet poor plasma). According to the present embodiment, SAGM is used as the preservation solution. Instead of SAGM, for example, MAP, OPTISOL (registered trademark) or the like may be used as the preservation solution.

The blood collection and blood returning unit 22 is connected to the ACD solution bag 24a via the connector 32 and a tube 40, together with being connected to the first cassette 24 via the connector 32 and two tubes 41 and 42.

The blood treatment unit 16 is capable of being attached to the centrifuge unit 18 (centrifugal rotor 18a) of the centrifugal separation device 14, and is configured in the form of a container in which blood can be introduced therein, flow therethrough, and flow out therefrom. The blood treatment unit 16 is connected to the first cassette 24 via a tube 43. The blood that has flowed out from the first cassette 24 flows into the blood treatment unit 16 via the tube 43. The blood treatment unit 16 is connected to the second cassette 26 via three tubes 44, 45, and 46. The blood components that have flowed out from the blood treatment unit 16 flow into the second cassette 26 via the tubes 44, 45, and 46.

The first cassette 24 comprises a sheet-shaped first cassette main body 25 made of a soft material. The first cassette main body 25 includes a blood collection line 34 through which blood collected from the donor flows, a rinsing solution line 35 connected to the blood collection line 34, and a blood returning line 36 through which blood components that are returned to the donor flow. The blood collection line 34 and the blood returning line 36 are flow paths which are independent of each other. The blood collection line 34 communicates with the lumens of the two tubes 41 and 43. The rinsing solution line 35 communicates with the lumen of the tube 54. The blood returning line 36 communicates with the lumens of the two tubes 42 and 53.

The first cassette main body 25 is made up of two sheets 25a and 25b made of a soft material, and the two sheets 25a and 25b are stacked in the thickness direction and are joined to each other by thermal fusion bonding or the like.

The first cassette 24 includes a convexly shaped flow path forming member 3B that forms the flow path (the blood collection line 34, the rinsing solution line 35, and the blood returning line 36). Even if there is no positive pressure acting within the flow path, the flow path forming member 38 bulges in convex shapes in the thickness direction of the first cassette 24 on both side surfaces of the first cassette main body 25. When pressed by an external force, the flow path forming member 38 can be elastically deformed in directions to close the flow path at the pressed locations thereof. More specifically, the flow path forming member 38 includes a blood collection line forming member 38a that forms the blood collection line 34, a rinsing solution line forming member 38b that forms the rinsing solution line 35, and a blood returning line forming member 38c that forms the blood returning line 36.

A filter accommodating unit 58 is provided in the blood returning line forming member 38c. A filter member 59 in the form of a sheet mesh is disposed inside the filter accommodating unit 58 for the purpose of removing clotted blood or blood clumps from the blood components that are returned to the donor.

In the first cassette main body 25, there are provided a first pressed portion 60 for measuring the internal pressure of the blood collection line 34, a second pressed portion 62 for measuring the internal pressure of the rinsing solution line 35, and a third pressed portion 64 for detecting the internal pressure of the blood returning line 36. The first pressed portion 60 is provided in the blood collection line forming member 38a. The second pressed portion 62 is provided in the rinsing solution line forming member 38b. The third pressed portion 64 is provided in the blood returning line forming member 38c. The third pressed portion 64 is constituted by the filter accommodating unit 58.

In order to measure the internal pressure of the blood collection line 34 during operation of the centrifugal separation device 14 in a state in which the first cassette 24 is attached to the centrifugal separation device 14, the first pressed portion 60 is a site that is pressed by a later-described first load detecting unit 112a which is installed in the centrifugal separation device 14. In order to measure the internal pressure of the rinsing solution line 35 during operation of the centrifugal separation device 14 in a state in which the first cassette 24 is attached to the centrifugal separation device 14, the second pressed portion 62 is a site that is pressed by a later-described second load detecting unit 112b which is installed in the centrifugal separation device 14. In order to measure the internal pressure of the blood returning line 36 during operation of the centrifugal separation device 14 in a state in which the first cassette 24 is attached to the centrifugal separation device 14, the third pressed portion 64 is a site that is pressed by a later-described third load detecting unit 112c which is installed in the centrifugal separation device 14.

On the first cassette main body 25, there are provided clamp action members 66a and 66b on which clamps 80a and 80b which are respectively flow path opening/closing mechanisms provided in the centrifugal separation device 14 act. When the first cassette 24 is installed in the centrifugal separation device 14, the clamp action members 66a and 66b abut against or are placed in facing relation, respectively, to their corresponding clamps 80a and 80b. The clamp action member 66a is disposed in the blood collection line 34. The clamp action member 66b is disposed in the rinsing solution line forming member 38b.

The second cassette 26 comprises a sheet-shaped second cassette main body 27 made of a soft material. The second cassette main body 27 includes a first line 71 through which blood components that have flowed out from a first outflow port 17a of the blood treatment unit 16 flow, a second line 72 through which blood components that have flowed out from a second outflow port 17b of the blood treatment unit 16 flow, and a third line 73 through which blood components that have flowed out from a third outflow port 17c of the blood treatment unit 16 flow. The second cassette 26 further includes a first connecting line 74 connecting the first line 71 and the second line 72, a blood platelet line 75 that branches off from the second line 72 and through which blood platelets flow, and a second connecting line 76 connecting the second line 72 and the third line 73.

The first line 71 communicates with the lumen of the tube 44 that is connected to the first outflow port 17a, together with communicating with the lumen of a tube 50 that is connected to the third cassette 28. The second line 72 communicates with the lumen of the tube 45 that is connected to the second outflow port 17b, together with communicating with the lumen of a tube 47 that is connected to the reservoir 30. The third line 73 communicates with the lumen of the tube 46 that is connected to the third outflow port 17c, together with communicating with the lumen of a tube 49 that is connected to the PPP bag 24f. The blood platelet line 75 communicates with the lumen of a tube 48 that is connected to the blood platelet bag 24e.

The second cassette main body 27 is made up of two sheets 27a and 27b made of a soft material, and the two sheets 27a and 27b are stacked in the thickness direction and are joined to each other by thermal fusion bonding or the like.

The second cassette 26 includes a convexly shaped flow path forming member 78 that forms the flow path (the first line 71, the second line 72, the third line 73, the first connecting line 74, the blood platelet line 75, and the second connecting line 76). Even if there is no positive pressure acting within the flow path, the flow path forming member 78 bulges in convex shapes in the thickness direction of the second cassette 26 on both side surfaces of the second cassette main body 27. When pressed by an external force, the flow path forming member 78 can be elastically deformed in directions to close the flow path at the pressed locations thereof.

More specifically, the flow path forming member 78 includes a first line forming member 78a that forms the first line 71, a second line forming member 78b that forms the second line 72, a third line forming member 78c that forms the third line 73, a first connecting line forming member 78d that forms the first connecting line 74, a blood platelet line forming member 78e that forms the blood platelet line 75, and a second connecting line forming member 78f that forms the second connecting line 76.

On the second cassette main body 27, there are provided clamp action members 66c to 66h on which clamps 80c to 80h which are flow path opening/closing mechanisms provided in the centrifugal separation device 14 act. When the second cassette 26 is installed in the centrifugal separation device 14, the clamp action members 66c to 66h abut against or are placed in facing relation to their corresponding clamps 80c to 80h. The clamp action member 66c is provided in the first line forming member 78a. The clamp action member 66d is provided in the first connecting line forming member 78d. The clamp action member 66e is provided in the second line forming member 78b. The clamp action member 66f is provided in the blood platelet line forming member 78e. The clamp action member 66g is provided in the third line forming member 78c. The clamp action member 66h is provided in the second connecting line forming member 78f.

A first pump action member 82a made of a soft material and which is pressed by the first pump P1 of the centrifugal separation device 14 is provided in the second line forming member 78b. A second pump action member 82b made of a soft material and which is pressed by the second pump P2 of the centrifugal separation device 14 is provided in the third line forming member 78c.

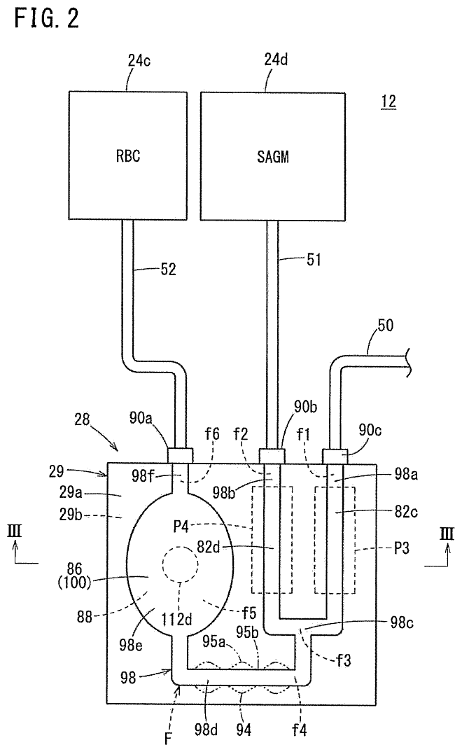

As shown in FIG. 2, the third cassette 28 comprises a sheet-shaped third cassette main body 29 having a flow path F formed in the interior thereof together with a filter accommodating unit 86, and a filter member 88 arranged inside the filter accommodating unit 86. The third cassette main body 29 is formed in a rectangular shape as viewed in plan. The third cassette main body 29 is formed of a soft material. For the soft material that constitutes the third cassette main body 29, the same material is used over the entirety of the third cassette main body 29. Moreover, the third cassette main body 29 may be constituted from a plurality of different materials.

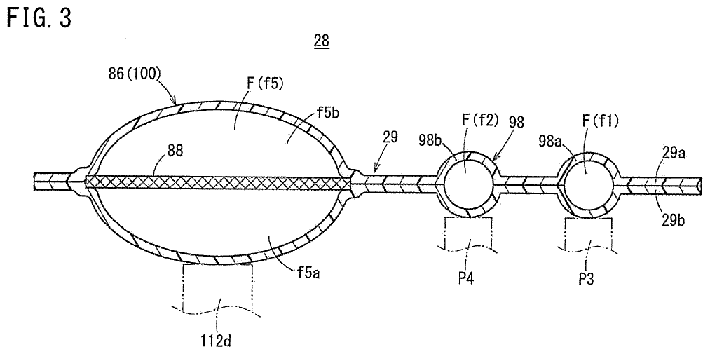

As shown in FIG. 3, the third cassette main body 29 includes a first sheet 29a and a second sheet 29b formed of a soft material. The first sheet 29a and the second sheet 29b are stacked in the thickness direction and are joined to each other. As examples of the soft material that constitutes the first sheet 29a and the second sheet 29b, there may be cited vinyl chloride, polyolefin, polyurethane, and the like. As examples of a plasticizer for vinyl chloride, there may be cited diisononylcyclohexane-1,2-dicarboxylate, bis-2-ethylhexyl phthalate, and the like.

The flow path F is formed between the first sheet 29a and the second sheet 29b. Fusion bonding (high frequency fusion bonding, thermal fusion bonding, etc.) is used as the means for joining the first sheet 29a and the second sheet 29b. The first sheet 29a and the second sheet 29b may also be joined together by another joining means (adhesion or the like).

As shown in FIG. 1, port members 90a to 90c, which are made of a hard material (for example, polypropylene, polycarbonate, or the like), are disposed on an outer peripheral edge portion of the third cassette main body 29. A tube 52 connecting the red blood cell bag 24c and the third cassette main body 29 is connected to the port member 90a. A tube 51 connecting the third cassette main body 29 and the preservation solution bag 24d is connected to the part member 90b. The tube 50 connecting the third cassette main body 29 and the second cassette main body 27 is connected to the port member 90c. The port members 90a to 90c are provided on the same side of the quadrangular shaped third cassette main body 29. The port members 90a to 90c may also be provided on different sides of the third cassette main body 29.

As shown in FIG. 2, the flow path F inside the third cassette main body 29 includes a red blood cell line f1 (blood component line) through which red blood cells flow, a preservation solution line f2 through which the preservation solution flows, a confluence section f3 in which the red blood cells and the preservation solution are made to merge together, a mixed liquid line f4 communicating with the confluence section f3 and through which a mixed liquid of the red blood cells and the preservation solution flows, a filter chamber f5 formed inside the filter accommodating unit 86 and which communicates with the mixed liquid line f4, and an outflow line f6 through which the mixed liquid after having passed through the filter chamber f5 flows toward the side of the red blood cell bag 24c.

The red blood cell line f1 and the preservation solution line f2 include portions that extend in parallel with each other. A portion of the red blood cell line f1 adjacent to the confluence section f3, a portion of the preservation solution line f2 adjacent to the confluence section f3, and the confluence section f3 are arranged on a straight line.

The third cassette main body 29 includes a convexly shaped flow path forming member 98 that forms the flow path (the red blood cell line f1, the preservation solution line f2, the confluence section f3, the mixed liquid line f4, the filter chamber f5, and the outflow line f6). As shown in FIG. 3, even if there is no positive pressure acting within the flow path F, the flow path forming member 98 bulges in convex shapes in the thickness direction of the third cassette 28 on both side surfaces of the third cassette main body 29. When pressed by an external force, the flow path forming member 98 can be elastically deformed in directions to close the flow path at the pressed locations thereof.

As shown in FIG. 2, the flow path forming member 98 includes a red blood cell line forming member 98a that forms the red blood cell line f1, a preservation solution line forming member 98b that forms the preservation solution line f2, a confluence section forming member 98c that forms the confluence section f3, a mixed liquid line forming member 98d that forms the mixed liquid line f4, a filter accommodating unit 86 serving as a filter chamber forming member 98e that forms the filter chamber f5, and an outflow line forming member 98f that forms the outflow line f6.

As shown by the imaginary line in FIG. 2, the mixed liquid line f4 may include a mixture promoting section 94 having a flow path cross-sectional area that varies along the direction of extension of the mixed liquid line f4. In this case, the mixed liquid line forming member 98d is configured in a manner so that each of a plurality of large diameter portions 95a (wide portions) and a plurality of small diameter portions 95b (narrow portions) are alternately arranged along the direction in which the mixed liquid line f4 extends, thereby forming the mixture promoting section 94 in which the flow path cross-sectional area thereof changes (increases or decreases).

A third pump action member 82c made of a soft material and which is pressed by the third pump P3 of the centrifugal separation device 14 is provided in the red blood cell line forming member 98a. A fourth pump action member 82d made of a soft material and which is pressed by the fourth pump P4 of the centrifugal separation device 14 is provided in the preservation solution line forming member 98b.

In the third cassette main body 29, there is provided a fourth pressed portion 100 for measuring the internal pressure of the filter chamber f5. The fourth pressed portion 100 is constituted by the filter accommodating unit 86. In order to measure the internal pressure of the filter chamber f5 during operation of the centrifugal separation device 14, in a state in which the third cassette 28 is attached to the centrifugal separation device 14, the fourth pressed portion 100 is a site that is pressed by a later-described fourth load detecting unit 112d which is installed in the centrifugal separation device 14.

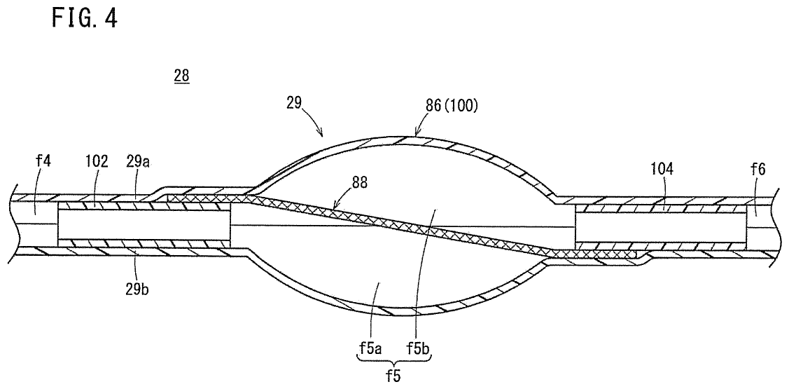

As shown in FIG. 4, the filter member 88 that removes (traps) white blood cells (the predetermined component) from the mixed liquid of red blood cells and the preservation solution is disposed in the filter accommodating unit 86. A first tube member 102 and a second tube member 104 are disposed on both sides of the filter accommodating unit 86. By the filter member 88, the filter chamber f5, which is an internal space of the filter accommodating unit 86, is partitioned into a first space f5a that communicates with the mixed liquid line f4 through the lumen of the first tube member 102, and a second space f5b that communicates with the outflow line f6 through the lumen of the second tube member 104.

The filter member 88 is made of a liquid permeable porous body having a large number of minute holes that communicate from one surface to the other surface thereof. As examples of the porous body, there may be cited a sponge sheet made of polyurethane, a nonwoven fabric, or the like. The filter member 88 may include a structure in which a plurality of sheet-shaped filter materials are stacked in the thickness direction.

As shown in FIG. 1, the reservoir 30 is connected to the second cassette 26 via the tube 47, together with being connected to the first cassette 24 via the tube 53. The interior of the reservoir 30 communicates with the second line 72 of the second cassette 26 via the lumen of the tube 47. The interior of the reservoir 30 also communicates with the blood returning line 36 of the first cassette 24 via the lumen of the tube 53.

Moreover, the flow path structures formed in the first cassette 24, the second cassette 26, and the third cassette 28, and the number and arrangement of the bags that are provided are not limited to the configurations shown and described above, but may be modified in accordance with the type of blood components to be collected, the method of use, and the like.

The centrifugal separation device 14 is a device that is used repeatedly during blood component collection, and is provided, for example, in a medical facility, a blood collection vehicle, or the like. The centrifugal separation device 14 is equipped with the centrifuge unit 18 (separation processing unit) having the centrifugal rotor 18a, and a cassette mounting unit 106 configured to enable attachment of the first cassette 24, the second cassette 26, and the third cassette 28 of the collection kit 12.

The cassette mounting unit 106 comprises an attachment base 108, a lid 110 that can be opened and closed and is configured in a manner so as to cover the attachment base 108 when closed, and clamps 80a to 80h which are configured to be capable of pressing respectively on the clamp action members 66a and 66b of the first cassette 24 and the clamp action members 66c to 66h of the second cassette 26. The cassette mounting unit 106 is equipped with a first load detecting unit 112a, a second load detecting unit 112b, and a third load detecting unit 112c, which are capable of pressing respectively on the first pressed portion 60, the second pressed portion 62, and the third pressed portion 64 provided in the first cassette 24. The cassette mounting unit 106 is further equipped with a fourth load detecting unit 112d which is capable of pressing on the fourth pressed portion 100 provided in the third cassette 28.

The lid 110, for example, is connected in a rotatable manner to the attachment base 108. When the lid 110 is closed in a state with the first cassette 24, the second cassette 26, and the third cassette 28 being held in predetermined positions (cassette mounting grooves) of the attachment base 108, the first cassette 24, the second cassette 26, and the third cassette 28 are sandwiched between the attachment base 108 and the lid 110.

The clamps 80a to 80h are capable of being advanced and retracted in the thickness direction of the first cassette 24 and the second cassette 26 that are in a state in which the cassettes are retained in the cassette mounting unit 106, and are disposed corresponding to the arrangement of the clamp action members 66a to 66h provided in the first cassette 24 and the second cassette 26.

At a time that the clamp action members 66a to 66h are not being pressed by the clamps 80a to 80h in a state in which the first cassette 24 and the second cassette 26 are mounted in the cassette mounting unit 106, the flow paths inside the clamp action members 66a to 66h are opened. When the clamps 80a to 80h press on the clamp action members 66a to 66h, the flow paths in the interior of the clamp action members 66a to 66h are closed. In addition, when the clamps 80a to 80h are retracted, due to the elastic restorative force of the clamp action members 66a to 66h, the clamp action members 66a to 66h are restored to their original shape, and the flow paths inside the clamp action members 66a to 66h are opened.

The centrifugal separation device 14 comprises an ACD solution transfer pump 114 which acts on the tube 40 for transferring the ACD solution, a blood collection pump 116 which acts on the tube 43 connecting the first cassette 24 and the blood treatment unit 16 and transferring blood therebetween, and a blood returning pump 118 which acts on the tube 53 connecting the reservoir 30 and the first cassette 24 and transferring the blood components that are returned to the donor.

The ACD solution transfer pump 114 is a pump that transfers the ACD solution from the ACD solution bag 24a to the blood treatment unit 16 via the first cassette 24, and is configured in a manner so as to cause the ACD solution to flow by pressing on the tube 40. The blood collection pump 116 is a pump that aspirates blood from the donor and feeds the blood into the blood treatment unit 16 via the first cassette 24, and is configured in a manner so as to cause the blood to flow by pressing on the tube 43. The blood returning pump 118 is a pump that returns the blood components, which are temporarily stored in the reservoir 30 after having flowed out from the third cassette 28, to the donor via the first cassette 24, and is configured in a manner so as to cause the blood components to flow by pressing on the tube 53.

The centrifugal separation device 14 is further equipped with the first pump P1 which acts on the first pump action member 82a provided in the second cassette 26, the second pump P2 which acts on the second pump action member 82b provided in the second cassette 26, the third pump P3 which acts on the third pump action member 82c provided in the third cassette 28, and the fourth pump P4 which acts on the fourth pump action member 82d provided in the third cassette 28.

The first pump P1 is a pump that feeds the blood components, which have flowed out from the second outflow port 17b of the blood treatment unit 16, into the reservoir 30 via the second cassette 26, and is configured in a manner so as to cause the blood components to flow by pressing on the first pump action member 82a. The second pump P2 is a pump that feeds the blood components, which have flowed out from the third outflow port 17c of the blood treatment unit 16, into the reservoir 30 or the PPP bag 24f via the second cassette 26, and is configured in a manner so as to cause the blood components to flow by pressing on the second pump action member 82b.

The third pump P3 is a pump that feeds the blood components (red blood cells), which have flowed out from the first outflow port 17a of the blood treatment unit 16, into the flow path F of the third cassette 28 via the second cassette 26, and is configured in a manner so as to cause the blood components to flow by pressing on the third pump action member 82c. The fourth pump P4 is a pump that causes the preservation solution to flow out from the preservation solution bag 24d, and guides the preservation solution to the flow path F of the third cassette 28, and is configured in a manner so as to cause the preservation solution to flow by pressing on the fourth pump action member 82d.

The ACD solution transfer pump 114, the blood collection pump 116, and the blood returning pump 118 are configured in the form of peristaltic pumps (so-called roller pumps). The ACD solution transfer pump 114, the blood collection pump 116, and the blood returning pump 118 may also be configured in the form of finger pumps. The first pump P1, the second pump P2, and the third pump P3 are configured in the form of finger pumps. The first pump P1, the second pump P2, and the third pump P3 may also be configured in the form of peristaltic pumps.

The centrifugal separation device 14 further includes a control unit 120. The control unit 120 includes a centrifuge control unit 122 for controlling the centrifuge unit 18, a clamp control unit 124 for controlling the clamps 80a to 80h, a pump control unit 126 for controlling the ACD solution transfer pump 114, the blood collection pump 116, the blood returning pump 118, the first pump P1, the second pump P2, the third pump P3, and the fourth pump P4, an internal pressure computation unit 128 that acquires (calculates) a circuit internal pressure of the collection kit 12, and a storage unit 130 in which predetermined information is stored.

Next, operations of the blood component collection system 10 according to the present embodiment, which is configured in the manner described above, will be explained.

As a preparation (set-up) for collecting blood components from the donor using the blood component collection system 10 shown in FIG. 1, the collection kit 12 is attached to the centrifugal separation device 14. More specifically, the first cassette 24, the second cassette 26, and the third cassette 28 are mounted in the cassette mounting unit 106, and the blood treatment unit 16 is attached to the centrifugal rotor 18a. On the other hand, the blood collecting needle 20 pierces and is inserted into the donor.

When the first cassette 24, the second cassette 26, and the third cassette 28 are mounted in the cassette mounting unit 106, at first, the third cassette 28 is mounted in the cassette mounting groove provided in the attachment base 108. In addition, by closing the lid 110, the first cassette 24, the second cassette 26, and the third cassette 28 are placed in a state of being held between the attachment base 108 and the lid 110. As a result, the first pressed portion 60, the second pressed portion 62, the third pressed portion 64, and the fourth pressed portion 100 of the collection kit 12 are placed in a state of being pressed respectively by the first load detecting unit 112a, the second load detecting unit 112b, the third load detecting unit 112c, and the fourth load detecting unit 112d of the centrifugal separation device 14. Further, the clamp action members 66a to 66h of the collection kit 12 assume a state of being placed in facing relation with respect to the plurality of clamps 80a to 80h of the centrifugal separation device 14.

When a command is issued by operation of a user with respect to the centrifugal separation device 14 in order to initiate operations, the centrifugal separation device 14 is operated roughly in the following manner. At first, in the centrifugal separation device 14, under an action of the ACD solution transfer pump 114, priming with the ACD solution is carried out.

Next, by rotating the centrifugal rotor 18a, the centrifugal separation device 14 applies a centrifugal force to the blood treatment unit 16 that is attached to the centrifugal rotor 18a, and together therewith, by operation of the second pump P2, blood (whole blood) from the donor is extracted and introduced into the blood treatment unit 16. By the centrifugal force that accompanies rotation of the centrifugal rotor 18a, the blood introduced into the blood treatment unit 16 is separated into red blood cells (concentrated red blood cells), blood platelets, and blood plasma.

After the red blood cells have been separated inside the blood treatment unit 16, the concentration the red blood cells is diluted by the preservation solution, the white blood cells are removed by the filter member 88, and the red blood cells are collected in the red blood cell bag 24c. The blood plasma that was separated inside the blood treatment unit 16 is collected in the PPP bag 24f. The blood platelets that were separated inside the blood treatment unit 16 are collected in the blood platelet bag 24e. During the centrifugal separation process and after the centrifugal separation process, the centrifugal separation device 14 returns to the donor the blood components that were temporarily stored in the reservoir 30. The blood collection operation to collect blood from the donor, and the blood returning operation to return blood components to the donor are repeated a plurality of times.

On the basis of the respective loads detected by the first load detecting unit 112a, the second load detecting unit 112b, the third load detecting unit 112c, and the fourth load detecting unit 112d, the centrifugal separation device 14 measures the internal pressures of the blood collection line 34, the rinsing solution line 35, the blood returning line 36, and the filter chamber f5, respectively. The circuit internal pressure of the respective sites is calculated by the internal pressure computation unit 128 of the control unit 120. The storage unit 130 stores a calibration curve that indicates a relationship between pressure values and the respective detected values (loads) of the first load detecting unit 112a, the second load detecting unit 112b, the third load detecting unit 112c, and the fourth load detecting unit 112d. The internal pressure computation unit 128 calculates the internal pressure with reference to the calibration curve.

The calculated (measured) circuit internal pressure, for example, ranges from −300 to 500 mmHg. The reference data used when calculating the circuit internal pressure using the loads detected by the first to fourth load detecting units 112a to 112d is not limited to a calibration curve, but may be a table that is prepared beforehand. In order to more accurately measure the circuit internal pressure, for example, another load detecting unit may further be provided in the centrifugal separation device 14, and the aforementioned calibration curve or the table may be corrected.

During operation of the blood component collection system 10, specifically, the centrifugal separation device 14 is operated in the following manner.

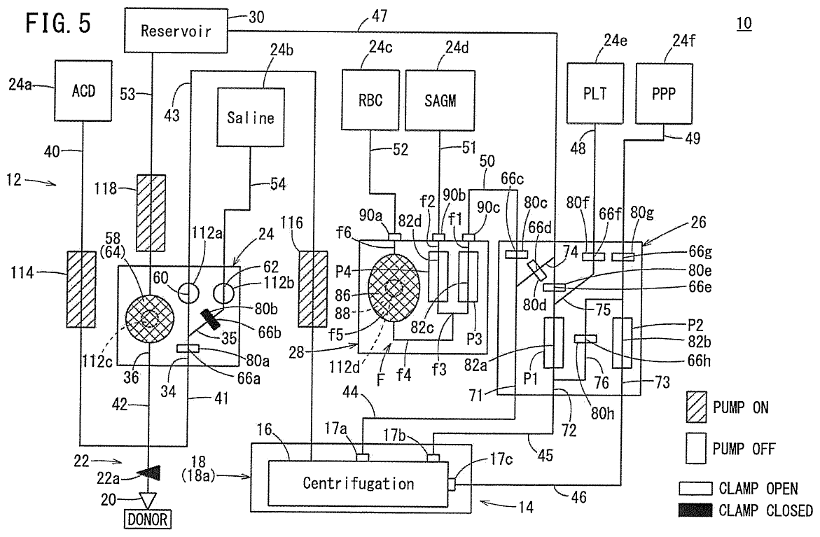

When priming with the ACD solution is carried out, as shown in FIG. 5, opened or closed states of the clamps 80a to 80h are set, and in this state, the ACD solution transfer pump 114, the blood collection pump 116, and the blood returning pump 118 are operated. The flow path of the blood collection and blood returning unit 22 is blocked by the clamp 22a. The ACD solution flows from the ACD solution bag 24a via the tube 40 into the two tubes 41 and 42 that are connected to the first cassette 24. At a stage at which it is detected by a non-illustrated line sensor outside of the first cassette 24 that the ACD solution has arrived in the immediate vicinity of the blood collection line 34 and the blood returning line 36, priming by the ACD solution is terminated.

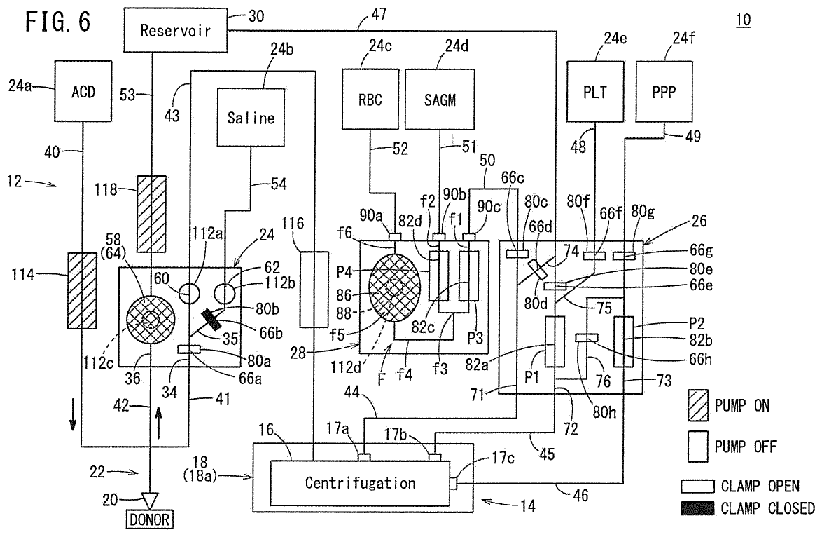

Next, in order to avoid air being returned to the donor at the time that the blood is returned, as shown in FIG. 6, the air inside the blood returning line 36 is removed. More specifically, as shown in FIG. 6, the opened or closed states of the clamps 80a to 80h are set, and the blood returning pump 118 is driven in a direction opposite to that during return of the blood, whereby the blood from the donor is guided into the tube 42, the blood returning line 36 of the first cassette 24, and the tube 53.

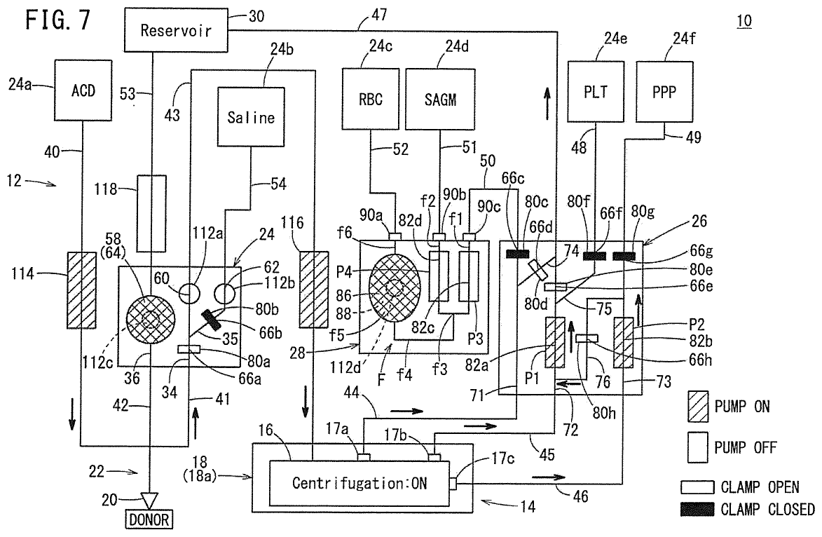

Next, as shown in FIG. 7, blood collection from the donor is started, and a centrifugation process in the blood treatment unit 16 is initiated. More specifically, as shown in FIG. 7, the opened or closed states of the clamps 80a to 80h are set, and by driving the blood collection pump 116, blood is introduced into the blood treatment unit 16 via the blood collection line 34 of the first cassette 24 and the tube 43. In this case, concurrently therewith, by driving the ACD solution transfer pump 114, the blood is introduced into the blood treatment unit 16 together with the ACD solution. In the blood treatment unit 16, the blood is separated by centrifugal force into layers depending on the specific gravity of the blood components.

The blood components (primarily red blood cells) that have flowed out from the first outflow port 17a are introduced into the reservoir 30 via the first line 71, the first connecting line 74, and the second line 72 of the second cassette 26. The blood components (primarily blood platelets) that have flowed out from the second outflow port 17b are introduced into the reservoir 30 via the second line 72 of the second cassette 26. The blood components (primarily blood plasma) that have flowed out from the third outflow port 17c are introduced into the reservoir 30 via the third line 73, the second connecting line 76, and the second line 72 of the second cassette 26. Introduction of the blood components that have flowed out of the blood treatment unit 16 into the reservoir 30 is continued until separation into layers of the blood in the blood treatment unit 16 has become stabilized to a certain extent (until a clear interface is formed to a certain extent between the layers of the separated blood components).

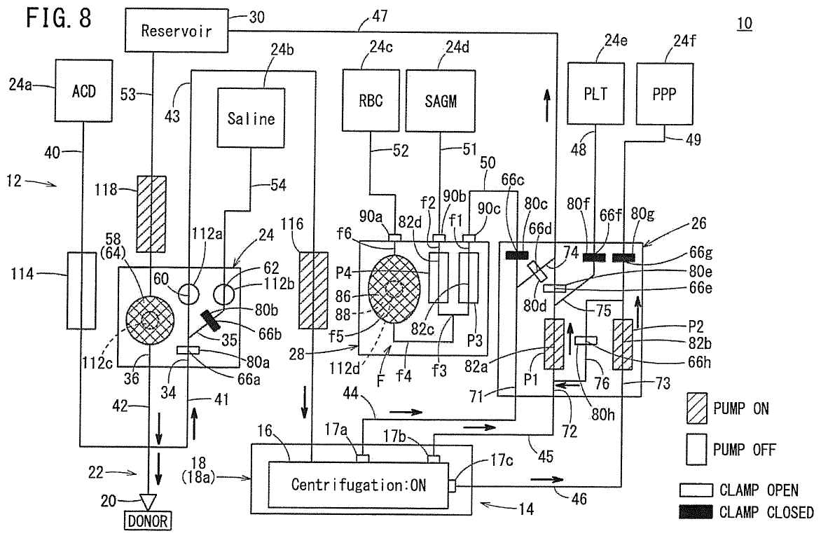

Next, as shown in FIG. 8, when a predetermined amount of the blood components has been stored in the reservoir 30, the process transitions to the blood returning operation. More specifically, the opened or closed states of the clamps 80a to 80h are set as shown in FIG. 8, and by driving the blood returning pump 118, the blood that was temporarily stored inside the reservoir 30 is returned to the donor via the blood returning line 36 of the first cassette 24. In this case, a portion of the blood from the reservoir 30 is introduced into the blood treatment unit 16 via the blood collection line 34 of the first cassette 24. The blood collection operation of FIG. 7 and the blood returning operation of FIG. 8 are repeated until separation into layers of the blood inside the blood treatment unit 16 has become stabilized.

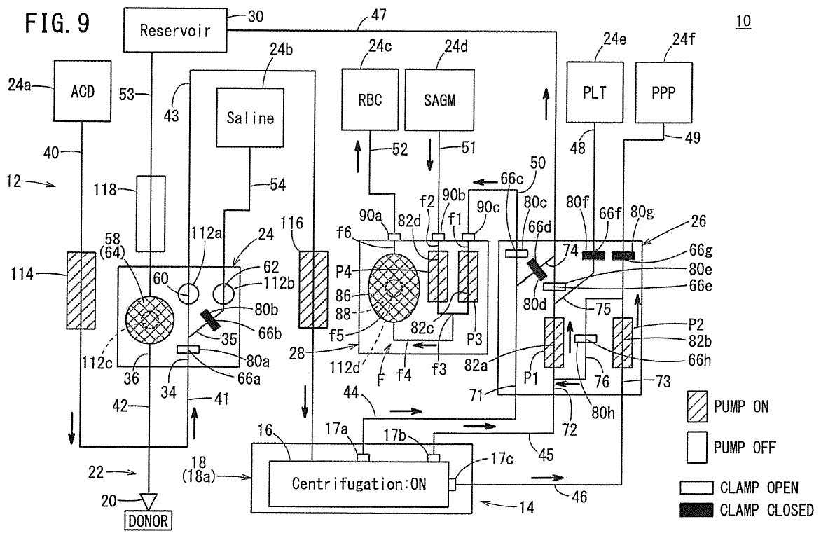

When separation into layers of the blood inside the blood treatment unit 16 is stabilized, then next, as shown in FIG. 9, the red blood cells are collected. More specifically, the opened or closed states of the clamps 80a to 80h are set as shown in FIG. 9, and by driving the third pump P3, the red blood cells (concentrated red blood cells) that have flowed out from the blood treatment unit 16 are introduced into the red blood cell line f1 of the third cassette 28, and together therewith, by driving the fourth pump P4, the preservation solution is introduced into the preservation solution line f2. In the third cassette 28, the red blood cells and the preservation solution are merged together in the confluence section f3 and become a mixed liquid (preservation-solution-added red blood cells). The preservation-solution-added red blood cells flow through the mixed liquid line f4 and flow into the filter chamber f5. The preservation-solution-added red blood cells are subjected to filtration during the process of passing through the filter member 88 disposed in the filter chamber f5, thereby becoming a formulation (leukocyte-removed red blood cells) from which white blood cells have been separated and removed, and the formulation is collected in the red blood cell bag 24c.

In order to efficiently separate and remove (filtrate) the white blood cells by the filter member 88, preferably, the viscosity of the preservation-solution-added red blood cells that are supplied to the filter member 88 is not too high. Accordingly, the hematocrit value (Hct), which is the proportion of red blood cells in the preservative-solution-added red blood cells, is preferably 60% to 70%, and more preferably, is on the order of 65% (64% to 66%). By controlling the third pump P3 and the fourth pump P4, and by respectively adjusting the flow rate of the red blood cells that flow to the red blood cell line f1, and the flow rate of the preservation solution that flows to the preservation solution line f2, the hematocrit value of the mixed liquid can be controlled to reside within a desired range.

During filtration by the filter member 88, the pump control unit 126 (see FIG. 1) controls the third pump P3 and the fourth pump P4 on the basis of the detection result of the fourth load detecting unit 112d, in a manner so that the pressure in the filter chamber f5 falls within a predetermined range. More specifically, on the basis of the detection result of the fourth load detecting unit 112d, the internal pressure computation unit 128 (see FIG. 1) calculates the internal pressure of the filter chamber f5. The pump control unit 126 controls the third pump P3 and the fourth pump P4 on the basis of the internal pressure of the filter chamber f5, in a manner so as to maintain as high a pressure as possible, within a range in which the internal pressure of the filter chamber f5 does not exceed a predetermined value.

The internal pressure of the filter chamber f5 varies depending on the flow velocity of the fluid flowing through the flow passage (the internal pressure also increases as the flow velocity increases). Accordingly, by controlling the third pump P3 and the fourth pump P4 and adjusting the flow velocity of the fluid flowing through the flow path, it is possible to control the internal pressure of the filter chamber f5. It was confirmed by experiment that the possibility of hemolysis (rupture or destruction of red blood cells) increases if the internal pressure of the filter chamber f5 exceeds 200 mmHg. Accordingly, it is preferable that the aforementioned predetermined value (upper limit threshold value of the internal pressure), for example, is 200 mmHg.

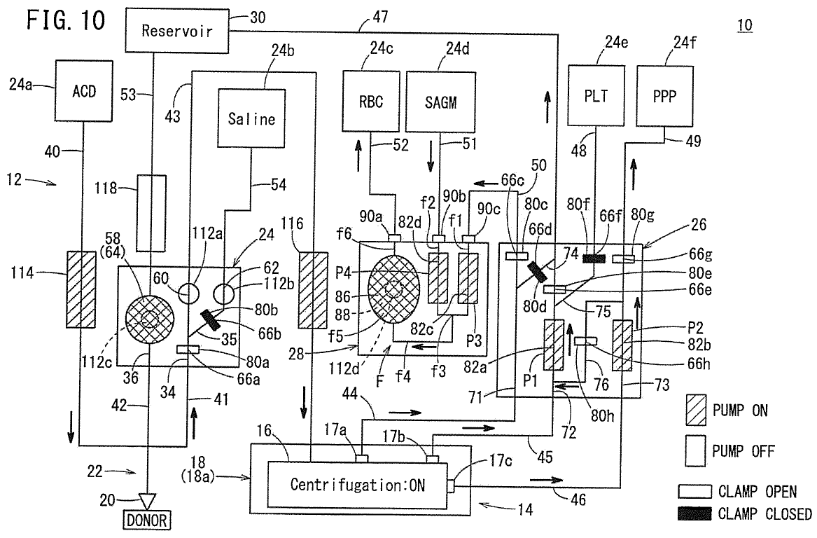

After having initiated the collection of red blood cells, when separation into layers inside the blood treatment unit 16 has become more stable (when the state of separation into layers has become suitable for the collection of blood plasma), then the blood plasma is collected as shown in FIG. 10. More specifically, the opened or closed states of the clamps 80a to 80h are set as shown in FIG. 10, and by driving the second pump P2, the blood plasma that has flowed out from the blood treatment unit 16 is transferred into the PPP bag 24f via the third line 73 of the second cassette 26. Concurrently therewith, by driving the first pump P1, the blood platelets that have flowed out from the blood treatment unit 16 are transferred into the reservoir 30 via the second line 72. When the stored amount inside the reservoir 30 has reached a predetermined amount, the blood returning pump 118 is driven, and blood is returned to the donor.

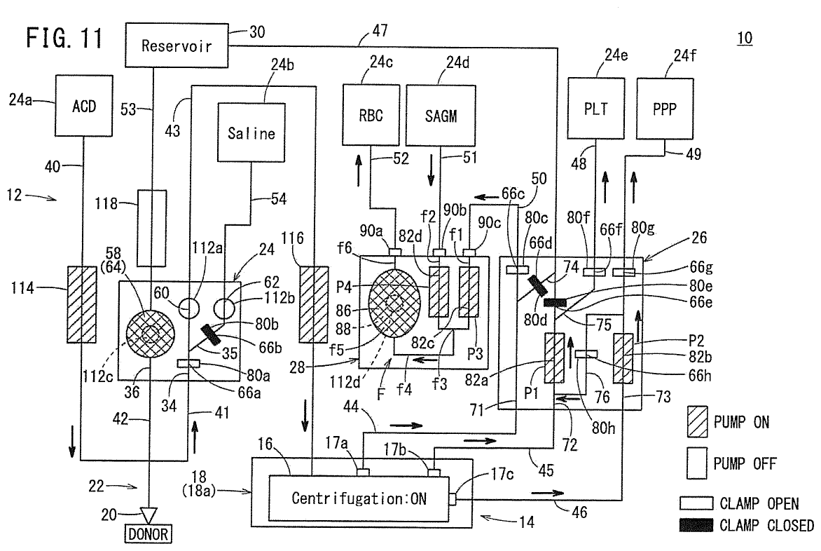

Next, as shown in FIG. 11, while the collection of red blood cells and blood plasma is continued, collection of blood platelets is also initiated. More specifically, the opened or closed states of the clamps 80a to 80h are set as shown in FIG. 11, and by driving the first pump P1, the blood platelets that have flowed out from the blood treatment unit 16 are transferred into the blood platelet bag 24e via the second line 72 and the blood platelet line 75 of the second cassette 26. During this period, transfer of blood components into the reservoir 30 is not carried out, and blood collection from the donor is continued.

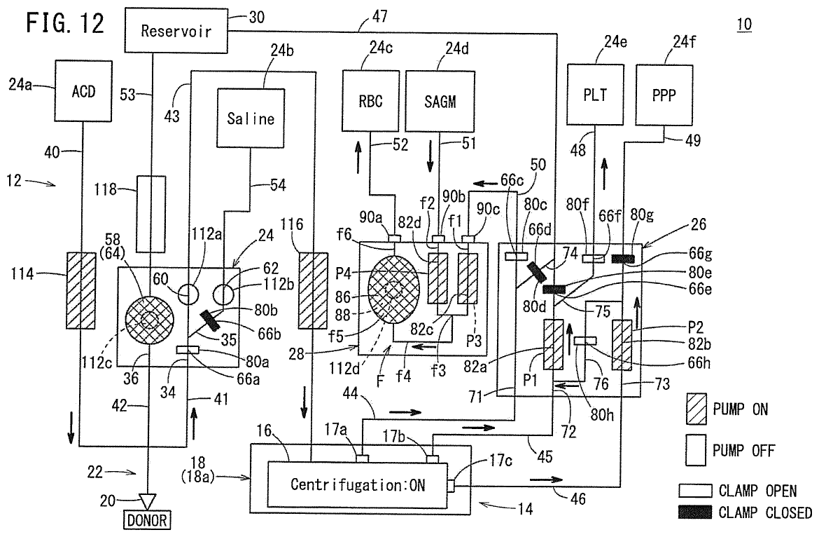

Next, as shown in FIG. 12, the blood platelet concentration is adjusted with blood plasma. More specifically, the opened or closed states of the clamps 80a to 80h are set as shown in FIG. 12, and by driving the second pump P2, and via the third line 73 and the second connecting line 76 of the second cassette 26, the blood plasma that has flowed out from the blood treatment unit 16 is made to merge with the blood platelets flowing through the second line 72. During this period, transfer of blood components into the reservoir 30 is not carried out, and blood collection from the donor is continued. In addition, a desired amount of red blood cells is collected in the red blood cell bag 24c, a desired amount of blood plasma is collected in the PPP bag 24f, and a desired amount of blood platelets is collected in the blood platelet bag 24e. Further, when the blood platelet concentration is adjusted to a predetermined concentration, the collection of blood components is brought to an end.

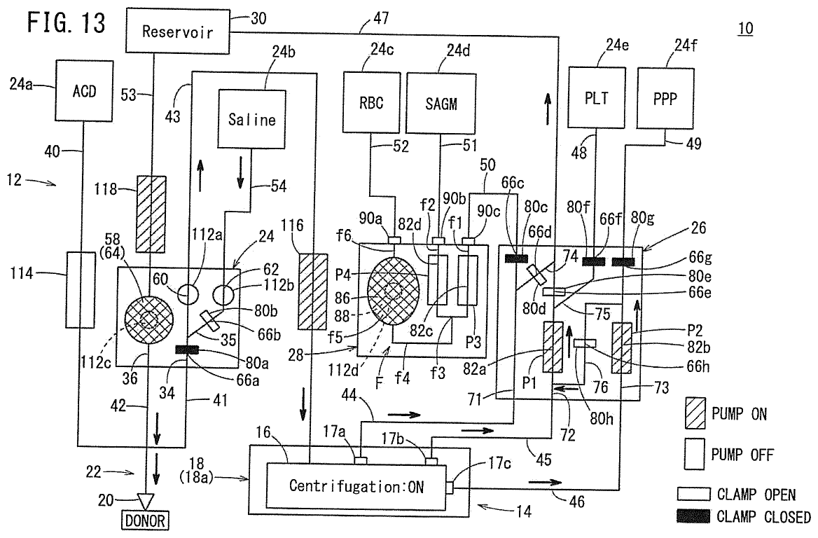

Next, as shown in FIG. 13, using physiological saline as a rinsing solution, the blood components in the circuit of the collection kit 12 are returned to the donor. More specifically, the opened or closed states of the clamps 80a to 80h are set as shown in FIG. 13, and by driving the blood collection pump 116, physiological saline is introduced into the blood treatment unit 16 via the blood collection line 34 of the first cassette 24 and the tube 43, and the blood components within that interval are pushed out. Further, by driving the first pump P1 and the second pump P2 in addition to the blood collection pump 116, the physiological saline that has flowed out from the blood treatment unit 16 is transferred into the reservoir 30 via the first line 71, the second line 72, the third line 73, the first connecting line 74, and the second connecting line 76 of the second cassette 26, and the blood components within that interval are pushed out. Furthermore, by driving the blood returning pump 118, the blood components of the blood returning line 36 are pushed out by the physiological saline and are returned to the donor.

In this case, the collection kit 12 and the blood component collection system 10 according to the present embodiment exhibit the following effects.

In accordance with the collection kit 12, the red blood cells and the preservation solution are mixed inside the third cassette main body 29, and when the mixed liquid passes through the filter member 88, the predetermined component is removed from the mixed liquid, and red blood cells from which leukocytes have been removed is obtained. In accordance with such a blood component collection kit 12, the filter which is made up from the filter member 88 and the filter accommodating unit 86 is formed integrally with the third cassette main body 29. Therefore, manufacturing costs of the blood component collection kit 12 can be reduced. Further, since the filter is set at a predetermined position accompanying attachment of the centrifugal separation device 14 to the collection kit 12, the operation of attaching the collection kit 12 to the centrifugal separation device 14 can be performed efficiently. Stated otherwise, the number of operations performed by the operator (a process to attach the filter structure) is reduced, and usability is enhanced.

In the flow path forming member 98 of the third cassette main body 29, there are provided the pump action members which are made of a soft material that are pressed by the pumps provided in the centrifugal separation device 14. In accordance with such a configuration, since the pump action members are provided integrally with the third cassette main body 29, it is possible to reduce manufacturing costs of a collection kit 12 provided with such pump action members, and together therewith, it is possible to efficiently carry out the operation of attaching the collection kit 12 with respect to the centrifugal separation device 14 equipped with the pumps.

The third pump action member 82c and the fourth pump action member 82d are provided respectively in the red blood cell line forming member 98a and the preservation solution line forming member 98b. In accordance with such a configuration, by individually controlling the flow rates of the red blood cells and the preservation solution, it is possible to adjust the concentration of the blood component in the mixed liquid to a desired range that is suitable for removal of the predetermined component by the filter member 88.

A portion of the red blood cell line f1 adjacent to the confluence section f3, a portion of the preservation solution line f2 adjacent to the confluence section f3, and the confluence section f3 are arranged on a straight line. In accordance with such a configuration, the red blood cells and the preservation solution collide head on at the confluence section f3. Therefore, mixing of the red blood cells and the preservation solution is promoted, and removal of the predetermined component can be efficiently performed by the filter member 88.

In the case that the mixed liquid line f4 includes the mixture promoting section 94 the flow passage cross-sectional area of which varies along the direction of extension of the mixed liquid line f4, mixing of the red blood cells and the preservation solution is promoted, and removal of the predetermined component can be efficiently performed by the filter member 88.

The filter accommodating unit 86 is constituted by the fourth pressed portion 100 made of a soft material and which is pressed by the fourth load detecting unit 112d provided in the centrifugal separation device 14 in order to detect the pressure of the filter chamber f5. Since the filter accommodating unit 86 serves in a dual manner as the fourth pressed portion 100 for detecting the pressure, streamlining of the structure can be achieved.

The pump control unit 126 controls the third pump P3 and the fourth pump P4 on the basis of the detection result of the fourth load detecting unit 112d, in a manner so that the pressure in the filter chamber f5 falls within a predetermined range. In accordance with such a configuration, while suppressing damage from occurring to the red blood cells which may be caused by excessive pressure, and by maintaining the pressure to be greater than or equal to a predetermined pressure, the ability to remove leukocytes can be enhanced, and the process can be carried out efficiently.

In the above-described third cassette 28, the flow path F is formed between the first sheet 29a and the second sheet 29b, which are formed of a soft material, however, the structure that forms the flow path F is not necessarily limited to such a configuration. For example, within the third cassette main body 29, the members that form the flow path F may be a plurality of tubes, and a plate shaped cassette base portion may be provided that supports the plurality of tubes. In this case, the cassette base portion is formed so that the filter accommodating unit 86 is exposed, in a manner so that the fourth load detecting unit 112d can be placed in contact with the filter accommodating unit 86.

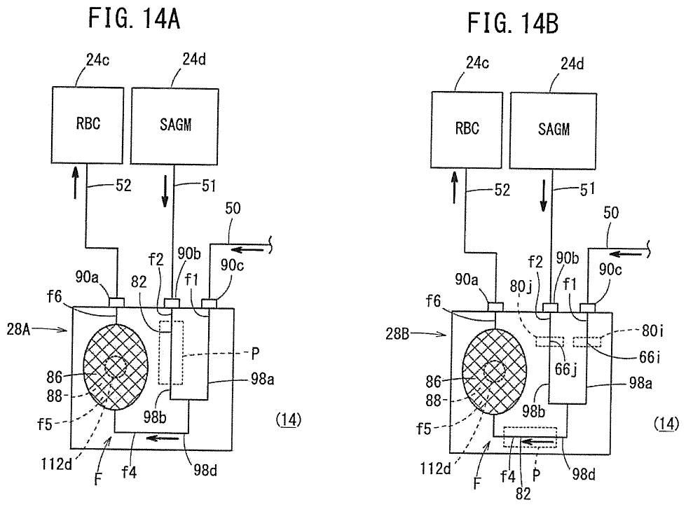

In a third cassette 28A according to another embodiment, as shown in FIG. 14A, from among the red blood cell line forming member 98a and the preservation solution line forming member 98b, a pump action member 82 is disposed only in the preservation solution line forming member 98b. The centrifugal separation device 14 is equipped with a pump P (which is the same as the above-described fourth pump P4) which acts only on the preservation solution line forming member 98b from among the red blood cell line forming member 98a and the preservation solution line forming member 98b. In accordance with such a configuration, since the number of pumps provided in the centrifugal separation device 14 is reduced, the configuration of the centrifugal separation device 14 can be simplified and costs can be reduced. In this case, the amount at which red blood cells flow into the third cassette 28A can be controlled by the blood collection speed or the like.

In a third cassette 28B according to yet another embodiment, as shown in FIG. 14B, the mixed liquid line forming member 98d is provided with a pump action member 82. Further, clamp action members 66i and 66j are provided respectively in the red blood cell line forming member 98a and the preservation solution line forming member 98b. The centrifugal separation device 14 includes a pump P that acts on the mixed liquid line forming member 98d. The centrifugal separation device 14 comprises clamps 80i and 80j that act respectively on the clamp action members 66i and 66j. In accordance with such a configuration as well, since the number of pumps provided in the centrifugal separation device 14 is reduced, the configuration of the centrifugal separation device 14 can be simplified and costs can be reduced. In this case, by setting the ratio between the flow path cross-sectional area of the red blood cell line f1 and the flow path cross-sectional area of the preservation solution line f2, it is possible to adjust the hematocrit value after confluence to a desired range.

The present invention is not limited to the above-described embodiments, and various modifications may be adopted within a range that does not depart from the essence and gist of the present invention.

1. A blood component collection kit configured to be attachable to a separation device adapted to separate blood components from blood, comprising:
a sheet-shaped cassette main body which is formed by stacking two sheets made of a soft material, and in which a flow path and a filter accommodating unit are formed between said two sheets; and
a filter member disposed inside the filter accommodating unit;
wherein the flow path includes
a blood component line in which the blood components flow,
a preservation solution line in which a preservation solution flows,
a confluence section in fluid communication with the blood component line and the preservation solution line in which the blood components and the preservation solution are made to merge together,
a mixed liquid line in fluid communication with the confluence section and through which a mixed liquid made up of the red blood cells and the preservation solution flows,
said filter accommodating unit in fluid communication with the mixed liquid line;
and a pump action member made of a soft material that is pressed by a pump provided in the separation device;
and
the pump action member is disposed in at least one of the blood component line and the preservation solution line.

2. The blood component collection kit according to claim 1, wherein:
from among the blood component line and the preservation solution line, the pump action member is disposed only in the preservation solution line.

3. The blood component collection kit according to claim 1, wherein:
the pump action member is disposed in the mixed liquid line.

4. The blood component collection kit according to claim 1, wherein a portion of the blood component line adjacent to the confluence section, a portion of the preservation solution line adjacent to the confluence section, and the confluence section are arranged on a straight line.

5. The blood component collection kit according to claim 1, wherein the mixed liquid line includes a mixture promoting section the flow passage cross-sectional area of which varies along a direction of extension of the mixed liquid line.

6. The blood component collection kit according to claim 1, wherein the filter accommodating unit comprises a pressed portion made of a soft material which is pressed by a load detecting unit provided in the separation device in order to detect a pressure of the filter chamber.

7. A blood component collection system equipped with a separation device adapted to separate blood components from blood, and a blood component collection kit configured to be attachable to the separation device;
wherein the blood component collection kit comprises:
a sheet-shaped cassette main body which is formed by stacking two sheets made of a soft material, and in which a flow path and a filter accommodating unit are formed between said two sheets; and
a filter member disposed inside the filter accommodating unit;
wherein the flow path includes
a blood component line in which the blood components flow,
a preservation solution line in which a preservation solution flows,
a confluence section in fluid communication with the blood component line and the preservation solution line in which the blood components and the preservation solution are made to merge together,
a mixed liquid line in fluid communication with the confluence section and through which a mixed liquid made up of the red blood cells and the preservation solution flows, and
said filter accommodating unit in fluid communication with the mixed liquid line;
and a pump action member made of a soft material that is pressed by a pump provided in the separation device;
and
the pump action member is disposed in at least one of the blood component line and the preservation solution line.

8. The blood component collection system according to claim 7, wherein:
the separation device comprises a load detecting unit, a pump, and a pump control unit adapted to control the pump;
the filter accommodating unit comprises a pressed portion made of a soft material which is pressed by the load detecting unit in order to detect a pressure of the filter chamber; and
the pump control unit controls the pump on the basis of a detection result of the load detecting unit, in a manner so that the pressure of the filter chamber falls within a predetermined range.
